(19) United States
(12) Patent Application Publication
(10) Pub. No.: US 2002/0116020 A1
(43) Pub. Date: Aug. 22, 2002

(54) THE CANNULA OF KURENKOV FOR CARRYING OUT OF THE OPERATION OF REFRACTIONAL CORRELATING EXIMER LASER INTRASTROMAL KERATECTOMY (REIK)

(76) Inventor: Vyacheslav Vladimirovich Kurenkov, Moscow (RU)

Correspondence Address: EPSTEIN, EDELL, SHAPIRO, FINNAN & LYTLE, LLC, 1901 RESEARCH BOULEVARD, SUITE 400, ROCKVILLE, MD, 20850, US

(21) Appl. No.: 09/958,059

(22) PCT Filed: Feb. 1, 2001
(86) PCT No.: PCT/RU01/00038

(30) Foreign Application Priority Data
Feb. 2, 2000 (RU) 2000102334
Jan. 31, 2001 (RU) 2001 102703

(51) Int. Cl. A61F 9/00

(52) U.S. Cl. 606/166

(57) ABSTRACT

The invention applies to the medical instruments and can be used during refractional correcting excimer laser intrastromal keratectomy (REIK) procedure. The cannula has a hollow body and curved working part having lateral canals and a cavity, corresponding with the cavity of the body. The lateral canals are made for instillation and/or lavage of interface during the refractional correcting excimer laser intrastromal keratectomy (REIK). The longitudinal axes of the lateral canals are situated across the plane of flexure of the working part and are oriented at an angle, or angles, to the longitudinal axis of the working part. The working part has a narrowing at an end thereof, for putting in and/or separation of tissues, and the body and/or the working part have identical cross-sections and/or cross-sections whose shapes are not the same.

### BACKGROUND OF THE INVENTION

1. Field of the Invention

This invention is in the field of ophthalmology and may be used during operations wherein correction of vision is carried out by an excimer laser. In particular, the present invention relates to medical instruments, and can be used in all types of surgery, in particular, at the operation of refractional correcting excimer laser intrastromal keratectomy (REIK) and other versions of intrastromal operations on a cornea.

2. Description of the Related Art

The cannulas are intended to carry out diagnostic and medical manipulations in the natural superficial cavities of the body and fistulous canals. They are produced mainly from metal, and like the needles, they are supplied with a mandrin.

In ophthalmology, cylindrical and flat cannulas are used to bathe the tears canal, to suck off lens mass, and also to put air and bathing liquids in the anterior chamber of the eye.

At present, the puncture needle is well known. It contains a cannula, an auxiliary tube and a working part, curved on the radius. There are some holes on the protuberant surface of the working part and the cut of the working part end is aimed in the direction of the protuberant surface. The puncture needle has a stop piece between the auxiliary tube and the working part (see patent of Russian Federation No 543395 cl. And 61 In 17/34, published Jan. 25, 1977).

Cannulas for carrying out of the laser intrastromal keratomileusis operation (LASIK) are known in the art. The cannula, used to bathe corneal shell of an eye, includes a source of bathing solution, a tube, which is moved manually and which has sufficient length for infiltration into the upper part of interface of the cornea caused by a surgical treatment. The end of the tube is a flattened head, on top of which a hole to put a bathing liquid is made, and also two holes are disposed from above and from below to put the fluid upwards and downwards. These top and bottom holes are situated under an angle 90 from the axis which is passing through the hole on the top and on the single straight line, in other case they can be displaced upwards or downwards (U.S. Pat. No. 5,755,700, cl. 604-257, published May 26, 1998).

The disadvantage of the above cannula is irregularity of delivering and distribution of the bathing liquid under a flap. As a result, some elements of the tissue, which was exposed by the excimer laser and some organic elements from conjunctival contents can be left under the flap.

### SUMMARY OF THE INVENTION

One of the main technical advantage of the Kurenkov design of cannula according to the present invention is a regular distribution of the bathing liquid under the flap and achievement of complete clearing of the upper part of interface. Also, due to creation of a stream of the bathing liquid in one direction, the possibility of mixing with the conjunctival liquid and organic elements is sharply decreased.

Such a result is reached in the cannula of Kurenkov, which has a hollow body and curved working part with sidelong canals and a cavity connected with the cavity of the body. Its lateral canals are executed for installation and/or lavage of interface during the operation of refractional correcting excimer laser intrastromal keratectomy (REIK). REIK includes carrying out of instillative anesthesia, sanation of conjunctival sac, installation of the vacuum ring, making a cut with formation of the flap on a hinge, turning aside of the flap, carrying out of laser ablation of the cornea, instillation of the medicinal preparations and repositioning of the flap. In particular, the anesthesia is carried out using a vasoconstrictor, and the vacuum ring is disposed so that its longitudinal axis passes through the center of the pupil, then the ring is moved vertically from top to bottom by 0.5-1.0 mm. Before formation of the flap a mixture of solutions of 0.4-0.6% of methylcellulose and 0.17-0.19% of a sodium hyaluronate is instilled in the conjunctival sac. The hinge of the flap is situated at 12 o'clock and the lower edge of the flap should be not more than 2 mm away from limbus. Before turning the flap aside, it is folded in half with internal surface inside, and after laser ablation the stromal bed is bathed by the agents, which improving regeneration, and after reposition, it is instillated by a solution of an antibiotic and non-steroid preparations. Then the flap is smoothed transpalpebrally manually so that the lateral axes of the cannula's lateral canals are situated across the plane of flexure of its working part and are oriented longitudinally at an angle, or angles, to the longitudinal axis of the working part. The working part has a narrowing at an end thereof for adding and/or separating tissues. The body and/or the working part of the cannula are executed with identical cross-sections and/or with cross-sections whose shapes do not coincide with each other.

The cannula has a bend of the working part which is about 130°-150°.

The cannula has longitudinal axes of sidelong canals directed under the angle 30°-50° to the longitudinal axis of the working part.

The cross-sections of the lateral canals of the cannula have one or more shapes, such as circles and/or ovals, and/or polygons.

The cannula is made from metal.

The cannula is used in the operation of refractional correcting excimer laser intrastromal keratectomy (REIK).

Summarizing, the cannula of the present invention has a hollow body and a curved working part with lateral canals and a cavity, corresponding with the cavity of the body. The lateral canals of the cavity are made for instillation and/or lavage of interface during refractional correcting excimer laser intrastromal keratectomy (REIK) procedure, including instillation anesthesia, sanation of conjunctival sac, installation of the vacuum ring, making a section with formation of the flap on a hinge, turning away of the flap, carrying out laser ablation of the cornea, instillation of the medicinal preparations and repositioning of the flap. In this case anesthesia is carried out with use of a vasoconstrictor, the vacuum ring is disposed so, that its longitudinal axis passes through the center of the pupil, then the ring is displaced vertically from top to bottom by 0.5-1.0 mm, before formation of the flap there are instilled in the conjunctival sac the mixtures of solutions of 0.4-0.6% of methylcellulose and 0.17-0.19% of hyaluronate of a sodium. Thus, the flap basis is situated at 12 o'clock and the lower edge of the flap should be not more than 2 mm away from the limbus. Before turning away of the flap it is folded in half the inner surface inside, after laser ablation of the cornea the stromal bed is lavaged by the agents improving regeneration, and after the reposition the solution of antibiotic and non-steroid drugs is instilled. Then the flap is smoothed transpalpebrally manually with the lateral axes of the lateral canals of the cannula situated across the plane of flexure of its working part and oriented longitudinally at an angle, or angles, to the longitudinal axis of the working part. The working part has a narrowing at an end thereof for putting in and/or separation of tissues, and the body and/or the working part have identical cross-sections and/or cross-sections whose shapes are not the same.

### BRIEF DESCRIPTION OF THE DRAWINGS

The present invention is described with reference to the following drawing figures:

FIG. 1 shows the cannula of Kurenkov according to the present invention (general view).

FIG. 2 shows the cannula of Kurenkov, wherein the holes situated closer to the end are made at an angle of 90°.

FIG. 3 shows the cannula of Kurenkov, wherein the working part is made with a radial bend.

FIG. 4 shows an example of the location of cannula of Kurenkov during bathing of the interface.

### DETAILED DESCRIPTION OF THE PREFERRED EMBODIMENTS OF THE PRESENT INVENTION

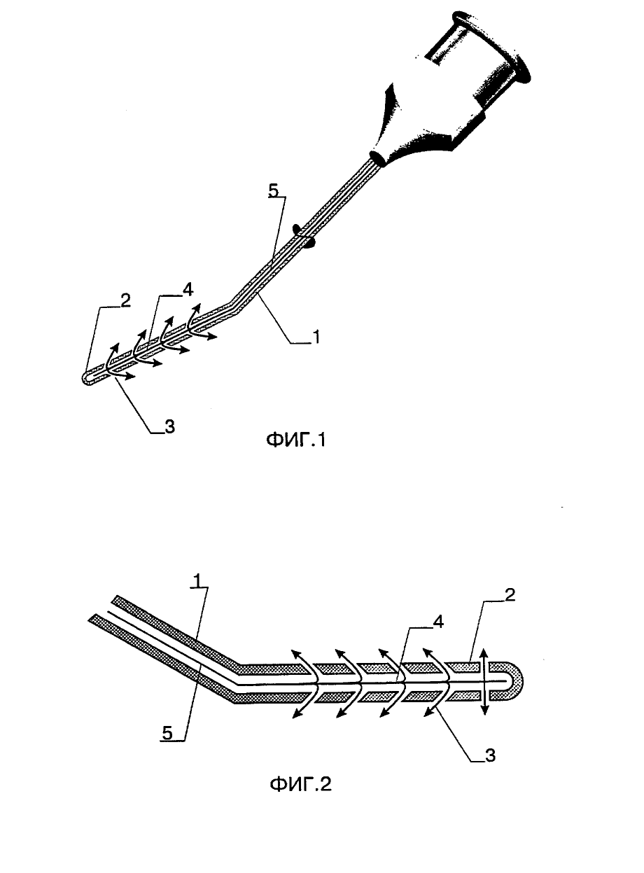

Referring to FIG. 1, the cannula of Kurenkov contains a hollow body (1) and a curved working part (2). In the working part there are made the lateral canals (3) and a cavity (4) which is connected with a cavity (5) of the body (1). The longitudinal axes of the lateral canals (3) are situated across the plane of a bend of the working part and are oriented at an angle, or under angles, to the longitudinal axis of a working part (2). The working part has a narrowing at an end thereof for insertion and/or separation of the tissues. The body (1) and/or the working part (2) are executed with identical cross-sections and/or with cross-sections, which do not have the same shape. As noted above, the cannula may be used during the refractional correcting excimer laser intrastromal keratectomy (REIK) and other versions of intrastromal operations on the cornea.

In an embodiment of the cannula of Kurenkov according to the present invention, as shown in FIG. 2, the holes which are closest to the end of the cannula, are made at an angle of 90°. Such design of the cannula can be used when the dimensions of the flap are small.

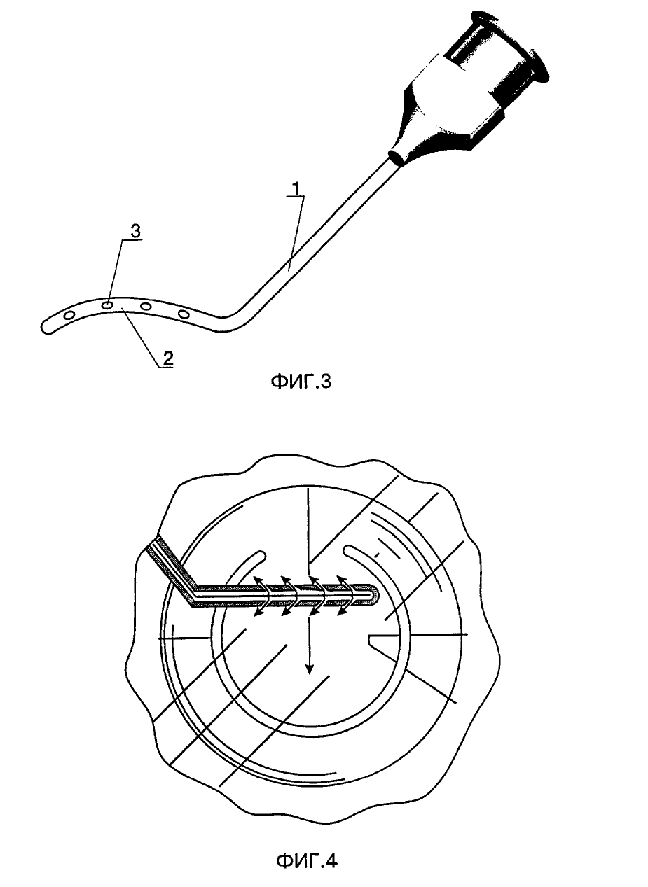

In another embodiment of the cannula according to the present invention, as shown in FIG. 3, the working part has a radial bend which allows to carry out lavage of the interface most painlessly.

The cannula according to the present invention can be used during refractional correcting excimer laser intrastromal keratectomy (REIK).

At present, in ophthalmology, argon-fluoride lasers having a wavelength of 193 nm are used for refractive surgery. The most advantageous of such laser is the Nidek-5000 2c. Its advantages include use of energy of small density, that allows to reduce trauma to the cornea and acoustic impact, besides temperature in the ablation zone practically does not increase. The system of beam delivery excludes formation of a central island, that frequently becomes a complication in case of use of wide beam lasers.

The method of the present invention is characterized by the following stages.

At the first stage for reception of the most predicted refractive result and reduction of its error, it is necessary to change the conditions of anesthesia of the cornea and by that to exclude or considerably reduce the damaging and toxic factors of anesthetic on an epithelium, and also to reduce its diffusion into the stroma. The decrease of diffusion will result in the reduction of influence of anesthetic on a hydration of the tissue. The change of permanent conditions of hydration normally influences the accuracy of work of excimer laser, as oedema of the corneal tissue interferes with the programmed removal of the tissue during one scan of the laser beam. However, the totality of the above requirements worsens the quality of the microkeratome cut because of less dense consistence of the stroma, increasing unevenness of the surface of the cut. The toxic property of anesthetic results in a maceration of epithelium, which influences the forthcoming adaptation of the flap because of a breach of the slip properties of the corneal surface.

To avoid or considerably decrease the specified unfavorable conditions it is offered to reduce the quantity of instillations of anesthetic by means of use of a vasoconstrictor, and, hence, the time till the beginning of the laser exposure and by that to impede increased hydration during operation. In turn, the time of action of the drop form of anesthetic together with the vasoconstrictor should not exceed 2-3 minutes prior to the start of the procedure, due to the time of a normal permeability of the cornea. In the method according to the present invention, Anesthetic is instilled 2-3 minutes before the beginning of laser exposure and that reduces the time of operation. Besides, the application of the vasoconstrictor allows to avoid a stage of drying of the stromal bed.

The second stage is lavage and sanation of the conjunctival sac with mechanical removal of foreign bodies and detritus. The sanation is carried out by the balanced isotonic solution lavage. The solution should be in an admixture with antibiotic, not just isotonic solution, as it is offered in the known method. The sanation of the conjunctival surface is carried out through the dispersing cannula by a syringe under pressure, that reduces the risk of infecting the interface after the operation.

The third stage is installation of the vacuum ring. The vacuum ring is installed on the cornea according to the center of a pupil, instead of the center of the cornea, with a vertical shift from top to bottom. The achievement of the optimal pressure in the eye is confirmed by the aplanative tonometer. The lower edge of the cornea flap should be not more, than 2 mm away from a limbus, as during physiological blinking, and, taking into account a standard position of the edge of the lower eyelid, the risk of shift of the flap after its reposition is reduced. In the late postoperative period there is a better fixation of the cornea flap to the stroma at such locating of the vacuum ring.

The fourth stage of the operation concerns intrastromal section with lubricant. According to an embodiment of the invention, the cut is effected only with use of a lubricant, namely an admixture of 0.4-0.6% of the methylcellulose solution and of 0.17-0.19% of the solution of a sodium hyalrunate. The chosen concentrations allow to reach maximum effect during sliding of the flap. Smaller concentrations make the solution thin, higher ones make it thick and do not produce the effect of sliding. The specified lubricant considerably reduces friction when the corneal flap is being cut out, and, hence, the possibility of damage of the epithelial surface of the flap is reduced also.

The fifth stage is separation and raising of the flap without contacts of the inner surface of the flap with air. This method means that a spatula is put under the flap base at 12 o'clock and the flap is moved parallel and upwards in the 12 o'clock direction. The inner surface of the flap thus does not contact air. The flap is laid at 12 o'clock and it is folded in half, inner surface inside. During sliding of the flap on the created intrastromal surface the fluid a very thin layer of the fluid is uniformly distributed around the whole area. In this case the amount of moisture in the section is optimal in order to create on the surface of the stromal bed a fluid layer which is a natural mask smoothing the section surface. The surface, built this way and leveled by the fluid, is optimal to carry out ablation of cornea.

In the method according to the present invention, after raising of the flap there is absent a stage of drainage of the surface by any method used in the known operation (by a scalpel, microsponge). Any touch of the surface is forbidden, as it can change its smoothness, which can result in the decrease of the quality of ablation of the cornea.

The sixth stage of the operation is a specialized photoablation of the cornea by the excimer laser. According to an embodiment of the present invention, only the excimer laser with slit-like rotating-scanning system of beam delivery can be used. It also requires the particular frequency of the beam and area of scanning, special nomograms (computer programs for work of the laser).

This type of laser excludes boiling of fluid on the surface, and prevents collection of excessive moisture in the central zone and formation of the central island. The speed of moving, angle of rotation and pulse frequency of the laser beam exclude excessive warming of the surface, as well as a burning of the tissue, and also promote more smooth and equal surface. This is an important advantage of the new method of operation.

The seventh stage of operation is processing of the ablated surface. The processing includes wetting and two stages of the mechanical cleaning. After the end of ablation the stromal surface is moistened with the balanced solution of a medicinal agent, improving process of regeneration of the cornea. In particular, Carnosine solution, pH of which corresponds to pH of the corneal tissue, may be used. Then remnants of the tissue elements on the ablated surface are removed by the corneal scraper. The movement should be done only in a vertical direction from 12 o'clock to 6 o'clock without a return in the opposite direction. If the movement of the scraper is made downwards-upwards or under any angle in relation to the flap hinge, the fluid will form on the surface of the stroma micro cylinders, that will affect unfavorably refractive results after operation.

The mechanical removal of micro elements should be finished by the lavage of the surface of the stromal section by antioxidants solution, for example, Carnosine, by means of the dispersing cannula by syringe under pressure. Thus the liquid layer is formed on the corneal surface.

The eighth stage is a reposition of the flap by the method of airless contact. The flap folded together is spread at 12 o'clock by moving from top to bottom uniformly on all surface. In the beginning, the inner surfaces slip relatively each other, then the free edge is moved by 6 o'clock. Because of sliding of the flap on the stromal surface, the layer of liquid rises on the surface of the corneal flap, eliminating the possibility of getting air and micro elements from the ambient air under a flap. Thus, an optimal reposition of the flap in its bed is achieved. This method minimizes an opportunity of getting undesirable inclusions under the flap. Thus, the time of the subsequent lavage of the interface is reduced and becomes less complicated and, hence, traumatic factor is minimized.

The ninth stage is lavage of the interface. The cannula is put under the flap basis at 12 o'clock. The corneal part of the cannula is situated parallel with the hinge of the flap, the end of the cannula should not reach the edge of the flap and, even more so, it should uplift it. When solution is delivered there is a flow of the fluid in one direction in the side of the uplifted slit-like canal formed around of the cannula body.

The tenth stage is instillation of the medicinal preparations. It is recommended immediately after the procedure of lavage is over to instill an antibacterial drug together with a non-steroid antiseptic and to remove the blepharostat at once. This action excludes a metabolic diffusion between the medical material and capillary layer of the interface fluid, taking into account possible osmolar processes. It enables to maintain a homogeneous thin layer, that has an immediate influence on uniform adaptation of the flap and the best sharpness of visual functions. On the other hand, a nonuniform layer interferes with optimal adaptation for various reasons.

The eleventh stage is a transpalpebral adaptation of the flap. After removal of the blepharostat the upper and lower eyelids are held by the fingers. Then, pressing by the finger on the upper eyelid and continuing to fix the lower one, one should move forcedly on the flap by the upper eyelid from 12 down to 6 o'clock. After this manipulation has been completed, the eyelid is released and it comes back on it's own. Then the patient is asked to blink, but one should continue to keep the lower eyelid fixed. Then the lower eyelid is released and the eye is observed for a duration of 1-2 minutes. This method allows to create the most uniform distribution of the residual interface fluid due to a uniform distribution of pressure around the whole area of the flap and because the excessive fluid from under it is displaced. The cannula according to the present invention can be used and for putting it into the tissue. One of the original advantages of this method of stacking is absence of the instruments and sponges. Due to that, the influence of disturbing factor is excluded, and the opportunity of improper adaptation is reduced to a minimum.

An example.

The patient K. has come to the clinic with the complaints of deterioration of vision. The medical examination exposed a myopia in the right eye −5.0 D. The operation of refractional correcting excimer laser intrastromal keratectomy (REIK) was offered to the patient and he agreed to it.

After the patient lies down on the operating table, the instillation of 0.5% solution Proparacaini is made. After 10-20 seconds of exposure a blepharostat was installed. The repeated instillation of 1% of Proparacaini solution was made at the position of the eye maximally upwards, downwards, directly, then 2 drops of the epinephrine of hydrochloride in dilution 1:1000 were instilled. After the exposition up to 60 seconds there was made a sanation of the conjunctival sac within 10-15 seconds by the admixture of the isotonic solution with cipromed solution. Then immediately the vacuum ring was installed according to the center of the pupil with a vertical shift downwards, and also taking into account, that the down edge of the flap after the cut was 1.5 mm from a limbus. A vacuum system was switched on, IOP was checked by the aplanation tonometer. There were instilled some drops of the admixture of 0.5% solution of methyl cellulose and 0.18% solution of hyaluronate of a sodium. Then the head of microkeratome assembled beforehand and the engine were installed on the pin of the vacuum ring. Then the flap was formed. After the vacuum system had been unsealed, the assembled system was removed. Moreover the eye of the patient remained co-axis with radial system of the laser. It was achieved by fixing a look of the patient on the aiming laser mark. After removal of the system a state of the cornea was visually estimated and a spatula was put under the flap near its hinge at 12 o'clock. Moving the spatula upwards in the plane of the cornea and in the direction of 12 o'clock the flap was moved and laid so that it was folded in half with the inner surface inside. The marks of the excimer laser were installed and centered with a visual axis and immediately ablation according to the nomogram made for this patient and injected in the computer of the laser before the operation began. After the end of ablation the surface of the cornea was moistened with the Carnasine solution. The oddments of microelements on the ablated surface were removed by spatula in the direction from 12 by 6 o'clock without moving back, then the surface was lavaged with the use of the dispersing cannula and a fluid layer was formed. Then the spatula was laid to the point of the bend of the flap and moved downwards to 6 o'clock so that the lower edge of the flap slipped on the stromal surface, and the fluid has remained on the epithelial surface. After this manipulation the flap was smoothed out completely. The next stage was placing the cannula under the flap basis so as to form the slit hole in the place where it was placed, while the opposite side remained close to the stroma. The solution was put in so that the cannula could move mainly in the direction of the slit hole, and the opposite side served a parameter of the pressure of the fluid circulating under the flap. It is slightly opened only when the pressure becomes excessive and serves as a signal for its reduction. The cannula was moved from 12 o'clock to 6 o'clock. The lavage procedure was controlled by a microscope. Then a solution of Cypromide and Dikhloph was instilled. After that the blepharostat was removed. The eyelids were fixed manually in the open position. The lower eyelid remained fixed, and the upper eyelid was slightly pressed and closed. Then the eyelid was released and it reverted on its own. Keeping the lower eyelid fixed, doctor asked the patient to blink. The position of the flap was visually estimated. Then the patient was asked to blink without keeping the lower eyelid fixed. After that the patient was lifted from the table. The first examination of the patient was made 2 hours after the operation and then he was observed within one year, and no changes of refraction have occurred.

The offered cannula of Kurenkov allows to carry out clearing of interface more effectively, as the whole interface is washed, the fluid flow is organized in one direction, and there are no zones in it, where the elements of tissue appearing after the action of excimer laser can remain.

What is claimed is:

1. A cannula has a hollow body and a curved working part with lateral canals and a cavity, corresponding with the cavity of the body; the lateral canals of the cavity are made for instillation and/or lavage of interface during refractional correcting excimer laser intrastromal keratectomy (REIK) procedure, including instillation anesthesia, sanation of conjunctival sac, installation of the vacuum ring, making a section with formation of the flap on a hinge, turning away of the flap, carrying out laser ablation of the cornea, instillation of the medicinal preparations and repositioning of the flap; in this case anesthesia is carried out with use of a vasoconstrictor, the vacuum ring is disposed so, that its longitudinal axis passes through the center of the pupil, then the ring is displaced vertically from top to bottom by 0.5-1.0 mm, before formation of the flap there are instilled in the conjunctival sac the mixtures of solutions of 0.4-0.6% of methylcellulose and 0.17-0.19% of hyaluronate of a sodium; thus, the flap basis is situated at 12 o'clock and the lower edge of the flap should be not more than 2 mm away from the limbus; before turning away of the flap it is folded in half the inner surface inside, after laser ablation of the cornea the stromal bed is lavaged by the agents improving regeneration, and after the reposition the solution of antibiotic and non-steroid drugs is instilled; then the flap is smoothed transpalpebrally manually with the lateral axes of the lateral canals of the cannula situated across the plane of flexure of its working part and oriented longitudinally at an angle, or angles, to the longitudinal axis of the working part; the working part has a narrowing at an end thereof for putting in and/or separation of tissues, and the body and/or the working part have identical cross-sections and/or cross-sections whose shapes are not the same.
